(19) United States
(12) Patent Application Publication
(10) Pub. No.: US 2011/0251230 A1
(43) Pub. Date: Oct. 13, 2011

(54) THERAPEUTICS FOR NEUROLOGICAL DISORDERS

(75) Inventors: Pamela Shaw, Yorkshire (GB); Richard Mead, Yorkshire (GB); Adrian Higginbottom, Yorkshire (GB); Sian Barber, Yorkshire (GB)

(73) Assignee: UNIVERSITY OF SHEFFIELD, Sheffield, Yorkshire (GB)

(21) Appl. No.: 13/125,097

(22) PCT Filed: Oct. 23, 2009
(86) PCT No.: PCT/GB2009/051432

§ 371 Date: Jun. 23, 2011

(30) Foreign Application Priority Data
Oct. 24, 2008 (GB) 0819530.7

(51) Int. Cl. A61K 31/435; A61P 25/00; A61K 31/341

(52) U.S. Cl. 514/289; 514/473

(57) ABSTRACT

The present invention relates to therapeutic compounds that are Nrf2-ARE pathway activators suitable for the treatment of diseases known to be mediated by oxidative stress such as motor neurone disease. The invention also includes compounds identified by methods of the invention for treatment of neurogenerative diseases.

The present invention relates to therapeutic agents for the treatment of neurological disorders known to be mediated by oxidative stress and in particular for the treatment of motor neurone disease and amyotrophic lateral sclerosis. The invention includes inter alia products for the treatment of neurological disorders.

### BACKGROUND

Oxidative stress refers to the cytopathologic consequences of a mismatch between the production of free-radicals and the ability of the cell to defend against them. Growing data from experimental models and human brain studies suggest that oxidative stress may play an important role in neuronal degenerative diseases. Oxidative stress has been implicated in both normal aging and in various neurodegenerative disorders such as Parkinson's disease, Alzheimer's disease and amyotrophic lateral sclerosis and may be a common mechanism underlying various forms of cell death including necrosis, apoptosis, and excitotoxicity.

Motor neurone disease (MND) is more commonly called Amyotrophic lateral sclerosis (ALS) in the USA and is a progressive, fatal, neurodegenerative disease characterised by the loss of motor neurones in the motor cortex, brain stem and spinal cord, this leads to weakness and atrophy. ALS typically leads to death within 2-3 years of diagnosis. ALS occurs in both sporadic (90% of all cases) and familial forms (10% of all cases). in 20% of familial ALS, mutations have been found in the copper, zinc superoxide dismutase gene (SOD1). The genes involved in the sporadic cases and in the remaining 80% of familial cases have yet to be identified. Currently there is no treatment which prevents or reverses the course of the disorder. Available treatments (such as riluzole and antioxidants) can at best only extend survival to a modest degree.

The mechanisms underlying the disease are not fully understood. However, the identification of SOD1 mutations as causative in a proportion of familial cases of ALS has allowed the generation of both cellular and mouse models of the disease which have greatly enhanced the understanding of disease mechanisms. Evidence from these models, as well as from patients, has provided very good evidence for a role of oxidative stress in disease pathogenesis. Oxidative stress has significant crosstalk to other potential mechanisms of neuronal injury, such as mitochondrial dysfunction, excitotoxicity, protein aggregation, cytoskeletal dysfunction and glial cell activation. It can feed into these mechanisms or is enhanced, in turn, by them. This central role in pathogenesis is reflected in a meta-analysis of studies in the most commonly used mouse model of ALS (transgenic mice expressing the G93A mutant form of human SOD1) where therapies targeting oxidative stress have been highlighted as demonstrating greatest promise in slowing disease progression.

Despite this central role, targeting oxidative stress in ALS has failed to translate into clinical benefit for patients, which may in part be due to the lack of sufficiently potent anti-oxidants able to access the CNS. A novel approach to limiting oxidative stress in neurodegenerative disease is to promote activation of the transcription factor, NFE2-related factor 2 (Nrf2). Nrf2 drives expression of a battery of Phase II detoxification and anti-oxidant enzymes via its interaction with the antioxidant response element (ARE). When activated, this ‘programmed cell life’ response is neuroprotective and conversely, attenuation of this pathway can enhance neuronal sensitivity to a range of neurotoxic challenges. Dysregulation of this pathway has been observed in ALS cellular models and confirmed in human tissue. Nrf2 itself and multiple target genes were repressed in a motor neuronal cell line expressing mutant (G93A) human SOD1. Further, a target of this pathway which plays an important role in mitochondrial anti-oxidant defence, peroxiredoxin3 (PRDX3), was downregulated in both this cellular model and human tissue from familial and sporadic ALS. G93A SOD1 transgenic mice, when crossed with an ARE reporter mouse, showed activation of the Nrf2-ARE pathway in muscle from 30 days of age, with marginal activation in spinal cord at 90 days and more robust activation at the 110 day time-point. At 90 days of age, mice have already begun to show muscle weakness and motor neuron loss and at 110 days they show significant motor neuron pathology. This suggests that activation of the Nrf2-ARE pathway in this murine model may be qualitatively insufficient or too late to protect motor neurons from significant damage. Taken in combination with other reports, this may reflect a defect in the capacity of cells expressing mutant SOD1 to activate this pathway.

The Nrf2-ARE pathway is an attractive therapeutic target in ALS because it is well defined, amenable to activation by small molecules and activation of cellular defences may confer a more lasting protection against oxidative stress than, for example a direct scavenging approach. It is known from the prior art that a flavonoid derived from green tea catechin-(−) epigallocatechin-3-gallate (EGCG) shows neuroprotective effects in a motor neuronal cell line expressing mutant (G93A) human SOD1. This cell line was partially protected from H₂O₂ induced cell death at concentrations of EGCG greater than 20 μM. This compound has also been tested in the G93A mouse model of ALS at a range of doses, once a day, orally from 60 days of age. At higher doses a significant extension in survival was seen with an increase in mean survival. This therapeutic effect is found in spite of the fact that EGCG is itself pro-oxidant, narrowing its therapeutic window, and highly polar, making it unlikely to cross the blood brain barrier in significant concentrations.

There is a need for therapeutic agents that are effective in the treatment of diseases known to be mediated by oxidative stress and especially diseases like motor neurone disease, ALS, Parkinson's and other neurodegenerative diseases.

There is a need for therapeutic agents for the treatment of neurodegenerative diseases that possess minimal toxicity and have the ability to cross the blood-brain barrier and so can penetrate the CNS.

### BRIEF SUMMARY OF THE DISCLOSURE

According to a first aspect of the invention there is provided a therapeutic agent for the treatment of motor neurone disease, the therapeutic agent being a Nrf2-ARE pathway activator selected from the group comprising andrographolide and S [+] apomorphine

Motor Neurone Disease (MND) is an all embracing term used to cover a number of illnesses of the motor neurone. Amyotrophic Lateral Sclerosis (ALS), Progressive Muscular Atrophy (PMA), Progressive Bulbar Palsy (PBP), Primary Lateral Sclerosis (PLS) are all subtypes. MND is the generic term used more in Europe whilst ALS is sometimes used more generically in the USA. It will be appreciated by the skilled person that references to motor neurone disease (MND) also extend to references to ALS, PMA, PBP and PLS and these named disease states may be used interchangeably.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The present invention has demonstrated through a cascade of screens and tests that andrographolide and S [+] apomorphine are potent and ‘drug-like’ Nrf2-ARE activators which also have the capacity to penetrate the CNS.

The compounds of the present invention may be used prophylactically or therapeutically either on their own or as part of a treatment regimen.

It will be appreciated that the term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relieve the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs.

According to a yet further aspect of the invention there is provided use of a Nrf2-ARE pathway activator selected from the group comprising andrographolide and S [+] apomorphine for the manufacture of a medicament for the treatment of motor neurone disease.

Apomorphine is a dopamine agonist typically administered subcutaneously as intermittent injections or in a continuous infusion. It is useful in managing advanced Parkinson's disease, and provides an alternative to neurosurgical procedures. There has been no previous clinical indication that [S]+ enantiomer of the compound may be useful in treating ALS or other disease states. Results have shown that in particular the S enantiomer which does not have dopamine agonist activity has the correct criteria for being an ALS therapeutic as defined by the methods of the present invention and as described hereinbefore. The present invention therefore recognises new therapeutic effects of the [S]+ enantiomer of apomorphine.

The chemical structure of apomorphine is given below and it will be appreciated that any variants and substitutions of the [S]+ enantiomer is encompassed within the compounds of the present invention. In addition any derivatives or salts of the [S]+ enantiomer are included within the scope of the present invention and the contents of PCT/US03108448 are incorporated herein by reference.

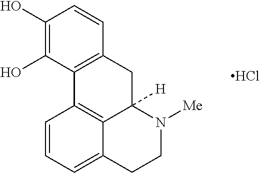

Andrographolide is a diterpenoid lactone of the plant Andrographis paniculata, known to possess anti-tumour activity for certain specific cancers such as breast cancer and to have an anti-inflammatory effect. There has been no previous clinical indication that the compound may be useful in treating diseases associated with oxidative stress for example and without limitation ALS. The present invention therefore recognises new therapeutic effects of andrographolide.

The chemical structure of andrographolide is given below and it will be appreciated that any variations and substitutions are encompassed within the compounds of the present invention. For example andrographolide and its derivatives may be of the formula where R.sub.1, R.sub.2 and R.sub.3 can independently represent hydrogen, acyl, phenyl, mono- or polyphosphate, mono- or polysulfate, glycosyl, cyclic or acyclic alkyl, alkenyl or alkynyl, wherein said phosphate or sulfate derivatives may be in the form of free acids or as salts.

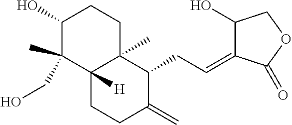

Preferably, the compounds of the present invention may be formulated into pharmaceutical forms suitable for administration to a patient in need of treatment.

The pharmaceutical compositions can be in any form suitable for oral, parenteral, topical, intranasal, ophthalmic, otic, rectal or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Accordingly the compounds of the present invention may further include pharmaceutical ingredients or excipients.

“Pharmaceutical ingredient” or “excipient” means a pharmacologically inactive pharmaceutically acceptable compound added to the compositions of the invention. The ingredient or excipient does not have any pharmacological properties.

According to a further aspect of the invention there is provided a therapeutic agent for the treatment of a neurodegenerative condition that occurs as a result of oxidative stress, the therapeutic agent being a Nrf2-ARE pathway activator selected from the group comprising andrographolide and S [+] apomorphine.

Preferably, the neurodegenerative condition know to be mediated by oxidative stress is selected from the group comprising motor neurone disease, amyotrophic lateral sclerosis (ALS) and primary lateral sclerosis (PLS), Huntington's disease, age related macular degeneration, preservation of organs for transplant/surgical procedures, stabilisation of cell cultures, photogenic oxidative stress and skin ageing and the treatment of radiation-induced cell damage.

According to a yet further aspect of the invention there is provided use of andrographolide for the treatment of a disease or condition that occurs as a result of oxidative stress, the disease or condition being selected from the group comprising motor neurone disease, amyotrophic lateral sclerosis (ALS), primary lateral sclerosis (PLS), Huntington's disease, Alzheimer's disease, Parkinson's disease, age related macular degeneration, preservation of organs for transplant/surgical procedures, stabilisation of cell cultures, photogenic oxidative stress and skin ageing and the treatment of radiation-induced cell damage.

According to a yet further aspect of the invention there is provided a method of treating an individual suffering from a neurodegenerative disease that occurs as a result of oxidative stress comprising administering a therapeutically effective amount of a Nrf2-ARE pathway activator selected from the group comprising andrographolide and S [+] apomorphine

According to a yet further aspect of the invention there is provided an in vitro method of screening a library of candidate therapeutic agents for their suitability to treat diseases know to be mediated by oxidative stress, the method comprising:
(i) exposing control or normal cells of non-neuronal origin to the candidate therapeutic and identifying candidate agents that possess an ability to activate the Nrf2-ARE pathway;
(ii) exposing cells of neuronal origin with candidate agents having a positive effect in step (i) and assessing their ability to activate the Nrf2-ARE pathway;
(iii) assessing the ability of the candidate agent to protect cells of neuronal origin against an oxidative stress insult; and
(iv) performing a series of in silico tests to ascertain physical and chemical parameters,

wherein a candidate therapeutic having positive results in step (i-iii) and having suitable physico-chemical properties is likely to be suitable for the treatment of diseases know to be mediated by oxidative stress.

The present invention provides a convenient cascade of tests as a screening method for identifying more potent and ‘drug-like’ Nrf2-ARE activators which also have the capacity to penetrate the CNS. The methods of the present invention provide a robust screening cascade to select a small number of promising molecules for further in vivo testing. These “hit” molecules are tested initially for their capacity to activate the Nrf2-ARE pathway in cell lines derived from normal non-human animals and cell lines of human origin and subsequently used as “tool” molecules to determine whether the pathway could be activated in neuronal cells derived by primary culture from the CNS of non-human animals.

Preferably, the non-human animal from which cells are derived are selected from the group comprising monkeys, dogs, cats, rabbits, rats and mice. Rodent species are preferred, and the mouse is the most preferred species.

Preferably, in one embodiment of the invention the cells are stably transfected with an ARE reporter construct, optionally the reporter is a fluorescent agent such as and without limitation GFP or the like.

Preferably, cells are assayed for increased expression of Nrf2 regulated genes. Suitable methods for detecting activation are described herein after.

Preferably, the cells of neuronal origin are CNS cells and especially are astrocytes. In one embodiment of the invention they are derived from transgenic non-human animals expressing the G93A mutant form of human SOD1.

Preferably, the oxidative stress test of step (iii) comprises withdrawal of serum and measurement of oxidative stress optionally with dichloroflourescein or derivatives or incubation with mitochondrial toxins (menadione) and measurement of cell survival. However, it will be appreciated that other stress test can be employed in the methods of the present invention.

Preferably, the suitable physical and chemical parameters of step (iv) are a cLogP _{<}5, molecular mass <500, <5 hydrogen bond donors (OH+NH count) and <10 hydrogen bond acceptors (O plus N atoms). The suitable physical and chemical parameters are typically referred to as Lipinski's rule of 5. In addition AlogP less than 4 but greater than 1, and a molecular Polar Surface Areas below 100 (ideally 80) are optimal for passive diffusion across the blood-brain barrier (BBB) however further preferred characteristics may also be applied to best select candidate therapeutic agents.

Any features ascribed to a particular aspect of the invention are intended to apply to each and every aspect mutatis mutandis.

### BRIEF DESCRIPTION OF THE DRAWINGS

FIG. 1 shows a schematic representation of the NRF2-ARE assay used in the present invention.

FIG. 2 shows the results of assay validation. FIG. 1a shows concentration response curves for tBHQ (open squares) and EGCG (closed squares) in the CHO-4×ARE-TK cell line. Both molecules have a narrow window of ARE activation peaking at 10 μM and 100 μM respectively. FIG. 1b shows results of Z′ score determination. The average +/− SD for vehicle (192 wells) and positive control (10 μM Ebselen, 192 wells) from a single 384 well plate are shown. The Z′ score for this assay was 0.51 with signal to noise (S/N) and signal to background (S/B) ratios of 12.8 and 2.9 respectively.

FIG. 3 shows example spectrum library screening data for one 384 well plate. GFP fluorescence versus well number. Wells 1-24 and 360-384 contain alternate positive (10 μM Ebselen) and negative (vehicle) controls. Dotted line represents the average +3SD of the negative controls and all compounds above this line are counted as ‘hits’. Some wells show reduced fluorescence, most likely due to toxicity.

FIG. 4a shows overlaid concentration response curves for all 46 hit compounds. Note that the profiles for many of the hit compounds follow a bell shaped dose response curve with reduced fluorescence due to toxicity at higher concentrations and have a narrow window of ARE induction. FIG. 4b shows examples of compounds with minimal toxicity at high concentrations or a broad window of ARE induction, note most of these compounds showed some toxicity at 100 μM.

FIG. 5 shows pharmacophore model and alignment of compounds from A/ Primary screen (compounds with EC50 <5 μM) and B/ Astrocytic oxidative stress assay in 1321N1 cells (compounds with EC50 <3 μM). Aromatic/Hydrophobic feature in green, Hydrogen Bond Acceptor feature in blue. For (B) a basic pharmacophore emerges with two hydrophobic features associated with a hydrogen bond acceptor. This is consistent with known NRF2 activators which may act by nucleophilic attack of sulphydryl groups on KEAP1, the cytoplasmic NRF2 regulator.

FIG. 6 shows the results of ARE reporter assay in C6 astrocyte cell line for 17 best hit compounds and S [+] apomorphine. Overall the response is similar to that seen in the CHO cell line and both R[−] and S[+] apomorphine induce the Nrf2-ARE pathway to a similar degree suggesting that this activity is unrelated to the dopamine agonist activity of R[−] apomorphine. The response in the NSC34 cell line was substantially reduced or non-existent for the majority of compounds (not shown).

FIG. 7 shows quantitative RT-PCR analysis for NRF2 regulated genes in the rat C6 astrocyte cell line (C6 astrocytes, FIG. 7a and FIG. 7b) and primary mouse astrocytes (FIG. 7c and FIG. 7d) following 24 h treatment with Andrographolide (Andro) and S[+] Apomorphine (Apo S) at EC₅₀ and EC₉₀ concentrations, determined in C6-4×ARE-TK reporter cells. Mutliplex PCR was used to interrogate expression levels of 9 genes of interest including Nrf2 itself following drug treatment in C6 cells. Only two genes, Haem oxygenase 1 and NQO1, showed statistically significant changes in gene expression, shown in (a) and (b) respectively. Standard quantitative RT-PCR of these two genes in primary mouse astrocytes under the same conditions were also performed (c) and (d). Asterisks indicate significant difference in DMSO control by one way ANOVA.

FIG. 8 shows NRF2 inducers protect motor neurones (MN) in primary mouse astrocyte/MN co-cultures from menadione stress. Co-cultures were pre-treated for 24 h with S[+] Apomorphine (Apo S) and Andrographolide (Andro) at their EC₅₀ and EC₉₀ concentrations respectively, as determined in rat C6 4×ARE-TK reporter cells. The co-cultures were then challenged for 6 hours with 10 μM menadione to induce oxidative stress. In DMSO control cells an approximately 25% reduction in motor neurone number was observed which was not seen in wells treated with either drug

FIG. 9 shows immunoflourescence staining for Haem Oxygenase 1 in primary mouse astrocytes following treatment with Nrf2 Inducers at EC₅₀ and EC₉₀ concentrations. Area and staining intensity were quantified using Image J and used to calculate a staining index.

FIG. 10 shows total Glutathione levels in primary mouse astrocytes (FIG. 10a) and in conditioned media collected from primary mouse astrocytes (FIG. 10b) following treatment with Nrf2 Inducers at EC₅₀ and EC₉₀ concentrations. Total glutathione levels were measured using standard methods following 24 h pre-treatment with Nrf2 inducers. All treatments significantly increased glutathione levels in astrocytes relative to DMSO control and the EC₉₀ concentrations of both drugs significantly increased extracellular glutathione levels (*p values <0.005). Data are average of three independent experiments

FIG. 11 shows quantitative RT-PCR analysis for the Nrf2 regulated genes NQO1 (a) and Haem oxygenase 1 (HOX1) in primary mouse astrocytes overexpressing G93A mutant SOD1 following 24 h treatment with Andrographolide (Andro) and S[+] Apomorphine (Apo S) at EC₅₀ and EC₉₀ concentrations. Asterisks indicate significant difference from DMSO control by one way ANOVA.

FIG. 12A shows the in vivo pharmacokinetic time course for S[+] Apomorphine (Apo S) and FIG. 12B shows the corresponding PK parameters in mice (n=3 or 4 per time point). FIG. 12C shows the QRT-PCR for HO-1 and NQO-1 in mice 6, 24 and 48 hours after a single subcutaneous injection of either 2.5 or 5.0 mg/kg S[+] Apomorphine (Apo S).

### DETAILED DESCRIPTION

Cell Culture

Chinese Hamster Ovary (CHO), NSC34 mouse motor neuronal cells, C6 (rat) and 1321N1 (human) astrocytic lines were routinely maintained in DMEM supplemented with 10% FBS and penicillin/streptomycin. The TK-EGFP reporter construct consists of a 123 bp thymidine kinase promoter inserted in the multiple cloning site of pEGFP (Clontech) and the ARE-TK-EGFP also contains four repeats of a 41 bp GST ARE motif (TAGCTTGGAAATGACATTGCTAATCGTGACAAAGCAACTTT) (SEQ ID NO:1) 3′ to the TK promoter. These plasmids were transfected into CHO, C6 and 1321N1 cell lines using Lipofectamine 2000 (Invitrogen) and following 10-14 days of selection in 0.5 mg/ml G418 they were expanded and selected for basal eGFP expression using fluorescence activated cell sorting (BD, FACSAria) with two sequential cell sorts for each cell line. These mixed populations of stable transfectants with basal eGFP expression were used in subsequent assays and designated 4×ARE-TK-GFP for the ARE containing line and TK-GFP for the control cell line. The NSC34 cells lines were transfected with G93A mutant SOD1 and stably transfected single cell clones were isolated by selection in 250 μg/ml G418 and cloning by limiting dilution.

ARE Reporter Assay—Spectrum Library Screen Validation

In order to screen the spectrum library of 2000 small molecule drugs and natural products the TK-GFP CHO ARE reporter cell line was subjected to a Z′ score assay in a 384 well plate (Greiner Bio-one, μClear, black) using a range of plating densities (5-20×10⁴/well plated 24 hours prior to assay) and different media. Alternate wells were incubated with 10 μM of Ebselen and vehicle (0.1% DMSO) for 24 hours followed by replacement of media with PBS containing 0.3 μM of ethidium homodimer-1 (EthD1). This concentration of Ebselen represents an approximate EC₉₀ for this drug. GFP fluorescence (ARE induction) was then measured at Ex₄₈₅ₙₘ/Em₅₃₀ₙₘ using a Fusion universal plate reader (Packard Bioscience). The z score was calculated as follows

$${Z}^{′} = \frac{1 - ( 3 {SD}^{*} + 3 {SD}^{-} )}{( {Ave}^{+} - {Ave}^{-} )}$$

where

SD⁺=standard deviation of positive control wells

3SD⁻=standard deviation of negative control wells

Ave⁺=Average fluorescence reading of positive control wells

Ave⁻=Average fluorescence reading of negative control wells

Signal to noise (S/N=Ave⁺/SD⁺) and signal to background (S/B=Ave⁺/Ave⁻) ratios were also determined for the different assay conditions. Acceptable Z′ scores were >0.5.

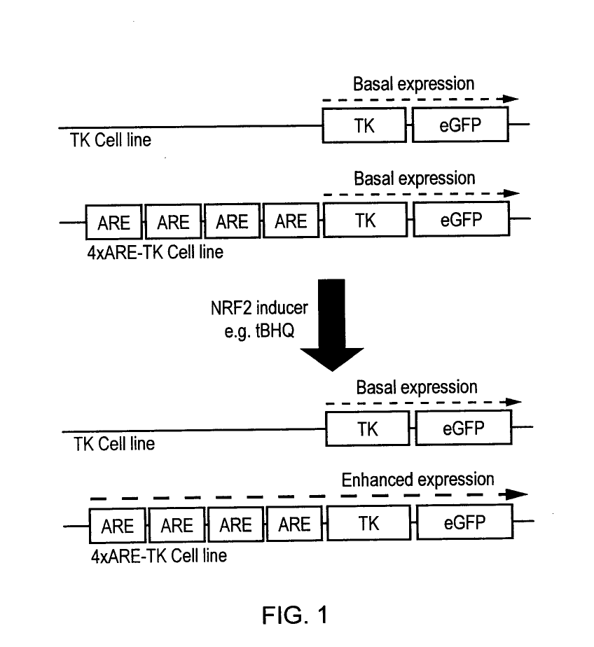

For the library screen, cells were plated at a density of 20×10⁴ in normal DMEM media containing 10% FBS on day −1 and on day 0, cells were incubated for 24 hours with drug in serum free media. Media was removed by hand and replaced (1 compound/well) with the Spectrum library diluted to 10 μM in 0.1% DMSO using a Q-bot liquid handling system (Genetix, New Milton, UK). The media was removed after 24 hours and replaced with the same volume of PBS containing 0.3 μM EthD1. GFP fluorescence (ARE induction, Ex₄₈₅ₙₘ/Em₅₃₀ₙₘ) and Eth D1 fluorescence (toxicity Ex_{530 nm}/Em_{645 nm}) were then measured using a Fusion universal plate reader (Packard Bioscience) The TK-GFP CHO ARE cell line was screened twice in a single point assay and the control TK-GFP CHO cell line was screened once to eliminate false positives. FIG. 1 shows a schematic representation of the NRF2-ARE assay.

ARE Reporter Assay—Determination of EC₅₀

Confluent cultures of 4×ARE-TK-GFP CHO or TK-GFP CHO expressing cells in 96 or 384-well tissue-culture plates were treated with drug (0.01-100□M in triplicate) or vehicle (0.1-1% DMSO) in FCS-free DMEM in triplicate for 24 hours. The media was removed and replaced with the same volume of PBS containing 0.3 μM EthD1. GFP fluorescence (ARE induction, Ex₄₈₅ₙₘ/Em₅₃₀ₙₘ) and Eth D1 fluorescence (toxicity Ex_{530 nm}/Em_{645 nm}) were then measured using a Fusion universal plate reader (Packard Bioscience). Non-linear regression was used to fit a sigmoidal dose-response curve on a semi-Log plot to calculate the EC₅₀ using GraphPad Prism (GraphPad Software). The reporter assay was conducted in a similar fashion in C6 and 1321N1 astrocyte cell lines stably transfected with the 4×ARE-TK-GFP and TK-GFP constructs except that Eth D1 was added directly in the media and read prior to washing the cells once and reading the DCF signal.

Oxidative Stress Assay

A simple oxidative stress assay was used to determine whether preconditioning with NRF2-ARE inducing drugs could protect against a subsequent oxidative stress insult (serum withdrawal). The NSC34, C6 and 1321N1 cells were plated in 96 well tissue culture plates to achieve 30% confluency. They were incubated with drug in triplicate wells as a 9 point titration (100□M to 10 nM) for 24 hours. Cell density was observed to ensure no significant toxicity or growth inhibition occurred. Media was then replaced with serum free, phenol free media for 5 hrs. Dichlorofluorescein (DCF) and ethidium homodimer (EthD1) (Molecular Probes, Paisley, UK) was added to the cells to a final concentration of 5 μM and 0.3 μM, and DCF and EthD1 fluorescence was read at Ex_{485 nm}/Em_{530 nm}, Ex_{530 nm}/Em_{645 nm} respectively after 1 hour. Cell survival assay was then performed on the cells as protection is measured as % reduction in DCF signal, therefore data points were excluded where a decrease in cell number was measured.

Cell Viability Assay

The method used was essentially as described previously in the art. Briefly methylthiazolyldiphenyl-tetrazolium bromide (MTT) was added to the cells and a blank well to a final concentration 0.5 mg/ml and incubated at 37° C. for 1-3 hrs depending on the cell line used. Cells and reaction product were solubilised in 20% SDS/50% DMF for 1 hr with shaking at room temperature before reading the absorbance at Ex_{695 nm}.

Basal Oxidative Stress Assay in G93A SOD1 Expressing NSC34 Cells (ALS Cellular Model)

The G93A SOD1 expressing NSC34 cells were plated in 96-well tissue-culture plates in phenol red-free DMEM containing 10% FBS until 30-40% confluent. They were then incubated with drug at 0.01, 0.1, 1, 1 μM or vehicle (0.1% DMSO) in triplicate wells for 24 hours. Cytosolic reactive oxygen species levels were measured using DCF and EthD1 as in the oxidative stress assay.

Primary Mouse Motor Neurone/Astrocyte Co-Cultures

Mouse glial cultures were established from C57BI/6 cortices from 1-2 day old pups. Cortices were dissected out and cells dissociated by trituration following incubation in DNasel, collagenase and trypsin. The incubation and trituration steps were repeated to ensure complete dissociation of cells. Cells were plated at 45,000 cells/cm² in DMEM containing 10% FBS, 100 U/ml penicillin, and 100 μg/ml streptomycin. After 24 hours, cultures were washed in PBS and grown for 2-3 weeks until confluent, changing the medium once a week. Confluent glial cultures were enriched for astrocytes by shaking and mild trypsinisation. Enriched astrocytes were grown to confluency and plated onto coverslips coated with poly-D-ornithine (1.5 mg/ml; Sigma, Poole, UK) at 40,000 cells/cm², and grown for 1 week.

Primary spinal cord motor neurons (MNs) were cultured from E13.5 wild-type C7BI/6 mouse embryos. Briefly, spinal chord preparations were dissociated by trituration following incubation in trypsin and DNasel. MNs were then isolated by density gradient centrifugation. Co-cultures were established by plating MNs at 8000/cm², on astrocytes, in Neurobasal medium supplemented with 1% B27, 2% horse serum, 50 mg/ml streptomycin, 50 U/mI penicillin, 0.5 mM L-glutamine, 25 mM glutamic acid (all from Invitrogen, Paisley, UK), 1 ng/ml BDNF, 10 ng/ml CNTF, and 100 pg/ml GDNF (all from R&D Systems, Abingdon, UK).

When co-cultures were established for 2 weeks, neuroprotection assays were performed by 24 hrs exposure to drug or vehicle followed by a 6 hour 10 micromolar menadione oxidative stress or glutamate. Following stress treatment, coverslips were washed 3 times, fixed and permeabilised to selectively stain motor neurons with SMI32 (Covance). Total MNs were counted by fluorescent microscopy in a 1.5 cm² area per coverslip. Minimum three repeats in triplicate were performed per condition. Both vehicle and drug treatments were counted before and after stress treatment, and results were statistically analysed by 2 way anova using Bonferroni post tests.

Total Glutathione Assay

Primary astrocytes were grown to confluency in 24 well plates and then treated with drug (or 0.05% DMSO vehicle) in phenol red-free DMEM containing 10% FBS and penicillin/streptomycin for 24 hours. Conditioned medium was collected and astrocytes were then washed in ice-cold PBS before addition of 250 μl/well of sulphosalicylic acid (SSA, 5% (w/v)). Plates were frozen at −80° C. and thawed at 37° C., twice, and then incubated at 4° C. for 15 minutes. The supernatant was removed and centrifuged at 13,000×g for 5 minutes. Conditioned medium samples were incubated at 80° C. for 15 minutes and then centrifuged at 13,000×g for 5 minutes. Samples were either used immediately or stored at −80° C. Reaction mixture (150 μl/well; 100 mM potassium phosphate buffer, pH 7.0, 1 mM EDTA, 6 Units/ml glutathione reductase, 1.5 mg/ml 5,5′-dithiobis(2-nitrobenzoic acid)) was added to 10 μl of each sample or glutathione standard (0-50 μM reduced glutathione) in a 96-well plate and incubated at room temperature for 5 minutes before addition of 50 μl/well of NADPH solution (0.16 mg/ml). A₄₁₂ₙₘ was measured every minute for 15 minutes and the total glutathione concentration (GSH+GSSG) was calculated from initial rates. Samples were tested in triplicate.

In Silico Analysis

In order to select drug-like molecules for further screening, Pipeline Pilot (SciTegic, London, UK) was used for in silico analysis. The molecular polar surface area (mPSA) was calculated for all 2000 molecules from the SPECTRUM collection as a crude measure of likely CNS penetrance {Ertl, 2000 #834} and a Lipinski Filter was also applied to determine which molecules were most drug-like. This filter applies the ‘rule of five’ which selects compounds with a cLogP <5, molecular mass <500, <5 hydrogen bond donors (OH+NH count) and <10 hydrogen bond acceptors (O plus N atoms).

Quantitative RT-PCR

Mutiplex PCr was used to detect changes in expression levels of target genes in the 1321N1 astrocyte cell line following treatment with Andrographolide and S(+) Apomorphine at EC₅₀ and EC₉₀ concentrations as determined in the NRF2-ARE reporter assay in C6 cells. The GenomeLab™ GeXP Genetic Analysis system (Beckman Coulter) was used to identify changes in gene expression in a multiplexed reaction for 9 genes of interest (Hmox1, Fth1, Keap1, Gclc, Nfe2I2, Gsr, Nqo1, Sqstm1 and EphX1) and three housekeeping genes (18s GAPDH and ACTS) using the following primers, outlined in the Table 1 below:

| Primer | Product | Left Primer Sequence | Left | Right Primer Sequence | Right |
|---|---|---|---|---|---|
| Name | Size | 5′-3′ | Tm | 5′-3′ | Tm |
| Hmox1 | 135 | AAGAGGCTAAGACCGCC | 59.983 | TGTCTGTGAGGGACTC | 60.299 |
| | | TTC | | TGGTC | |
| | | (SEQ ID NO: 2) | | (SEQ ID NO: 3) | |
| Fth1 | 152 | CCTGAATGAGCAGGTGA | 59.685 | CAGGGTGTGCTTGTCA | 59.873 |
| | | AATC | | AAGA | |
| | | (SEQ ID NO: 4) | | (SEQ ID NO: 5) | |
| Keap1 | 159 | ACGACGTGGAGACAGAG | 60.315 | AAAAGTGTGGCCATCG | 60.14 |
| | | ACC | | TAGC | |
| | | (SEQ ID NO:6) | | (SEQ ID NO: 7) | |
| Gclc | 173 | AAAGGCCTCTAAGCCAG | 59.848 | GCAGATAGTGGCCAAC | 60.142 |
| | | ACC | | TGGT | |
| | | (SEQ ID NO: 8) | | (SEQ ID NO: 9) | |
| Gapdh | 180 | AGACAGCCGCATCTTCT | 60.02 | TTGATGGCAACAATGTC | 60.376 |
| | | TGT | | CAC | |
| | | (SEQ ID NO: 10) | | (SEQ ID NO: 11) | |
| 18S | 192 | AAACGGCTACCACATCC | 59.993 | CCTCCAATGGATCCTC | 59.887 |
| | | AAG | | GTTA | |
| | | (SEQ ID NO: 12) | | (SEQ ID NO: 13) | |
| Nfe2l2 | 212 | CAGCATACAGCAGGACA | 60.287 | CAGTGAGGGGATCGAT | 60.072 |
| | | TGG | | GAGT | |
| | | (SEQ ID NO: 14) | | (SEQ ID NO: 15) | |
| Gsr | 241 | CGCCCATAGACTTTTTG | 59.702 | CATTTCGTCTTCCTCGT | 60.111 |
| | | AGG | | GGT | |
| | | (SEQ ID NO: 16) | | (SEQ ID NO: 17) | |
| Nqo1 | 251 | ACCTTGCTTTCCATCAC | 59.973 | TCTCCAGACGCTTCTTC | 60.533 |
| | | CAC | | CAC | |
| | | (SEQ ID NO: 18) | | (SEQ ID NO: 19) | |
| Actb | 273 | TTGCTGACAGGATGCAG | 60.136 | AAGGGTGTAAAACGCA | 59.383 |
| | | AAG | | GCTC | |
| | | (SEQ ID NO: 20) | | (SEQ ID NO: 21) | |
| Sqstm1 | 296 | TGGCCATGTCCTATGTG | 59.924 | AAGGGGTTGGGAAAGA | 60.298 |
| (v1 and | | AAA | | TGAG | |
| v2) | | (SEQ ID NO: 22) | | (SEQ ID NO: 23) | |
| Ephx1 (v1 | 349 | GGGCTTGACATCCACTT | 59.943 | CCCGCCTTGAATGTAG | 60.318 |
| and v2) | | CAT | | AATTT | |
| | | (SEQ ID NO: 24) | | (SEQ ID NO: 25) | |

Those shown in bold flank intronic sequences, preventing genomic background. The RT reaction mixture was assembled in a 96 well microplate as follows 3 μl DNase/RNase Free H2O, 4 μl RT Buffer 5×, 2 μl RT Rev Primer Plex Reverse Transcriptase, 5 μl KANr RNA, 5 μl Sample RNA (20 ng/μL) to give a total reaction volume of 20 μl. The samples were then incubated as follows, 48° C. 1 minute, 37° C. 5 minutes, 42° C. 60 minutes, 95° C. 5 minutes, 4° C. Hold. The PCR reaction mixture was assembled as follows in a 96-well sample microplate; 4.0 μl PCR Buffer 5×, 4.0 μl 25 mM MgCl2 (Abgene), 2.0 μl PCR Fwd Primer Plex, 0.7 μl Thermo-Start DNA Polymerase, (ABgene AB-0908/A), 9.3 μl cDNA Samples from RT reaction. The PCR was run as follows, (1) 95° C. 10 minutes, (2) 94° C. 30 seconds, (3) 55° C. 30 seconds, (4) 70° C. 1 minute, (5) Repeat steps 2-4 for an additional 34 cycles, (6) 4° C. Hold. On the basis of preliminary experiments reverse RT primer concentrations were optimised to allow detection of all products in a single reaction. PCR reaction products were diluted in SLS (sample loading solution) and separated on the GeXP capillary electrophoresis unit and the data analysed using the GeXP software and Microsoft Excel data package to give a fold change in expression relative to GAPDH and ACTB.

Standard QPCR

QPCR was carried out as previously described in the publication Wood-Allum et al 2005 with the essential difference that RNA was isolated from primary astrocytes or the astrocyte cell line using the RNeasy kit (Qiagen) following manufacturers' instructions. cDNA was prepared using Superscript II (Invitrogen) driven by oligo dT following manufacturers' guidelines. Quantitative RT-PCR. (Q-PCR) was performed using 25 ng of cDNA, 1× SYBR Green PCR Master Mix (Stratagene) and optimised concentrations of forward and reverse primers (Table 1), in a total volume of 20 μl and in triplicate for each sample. Following denaturation at 95° C. for 10 min, products were amplified by 40 cycles of 95° C. for 15 s and 60° C. for 1 min, on an MX3000P Real Time PCR System (Stratagene). To ensure amplification of a single product the dissociation curve was produced for each amplification. The relative levels of the genes of interest in the samples was determined by normalizing the level of expression to that of gapdh in each of the samples and by using the ddCt calculation (SYBR Green PCR mix and RT-PCR protocol, Applied Biosystems). Overall relative concentrations of the genes of interest were calculated following drug treatment by normalising the data to the cells grown in the vehicle (DMSO).

Statistical Analysis

For the Spectrum library screen, each 384 well plate contained 32 wells incubated with vehicle only (0.1% DMSO in media). The average fluorescence reading and standard deviation of these wells was calculated on a plate by plate basis. Hits were classified as having a GFP fluorescence value greater than the vehicle average plus three standard deviations. Toxicity was defined in the same way (i.e. EthD1 fluorescence value greater than the vehicle average plus three standard deviations).

### EXAMPLE 1

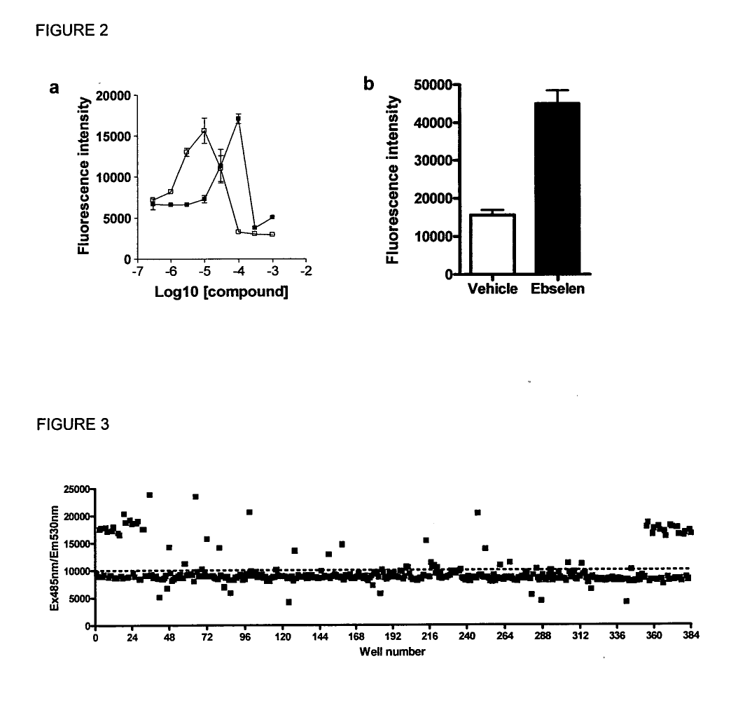

The 4×ARE-TK-GFP and TK-GFP reporter cell lines were tested for their response to the known ARE inducers tert-Butyl Hydroquinone (tBHQ) and the flavonoid EGCG at a range of concentrations. The compounds were applied in triplicate to confluent cells in 96 well plates in serum free medium for 24 hours and the induction of GFP measured in a fluorescence plate reader. Both compounds induced GFP expression in a narrow window, with EGCG peaking at 100 μM and tBHQ peaking at 10 μM (FIG. 2a). At concentrations higher than this peak expression, both compounds showed signs of toxicity by direct observation (cell loss) or increased Ethidium Homodimer fluorescence. No increase in fluorescence was seen in the control TK-GFP cell line (not shown).

### EXAMPLE 2

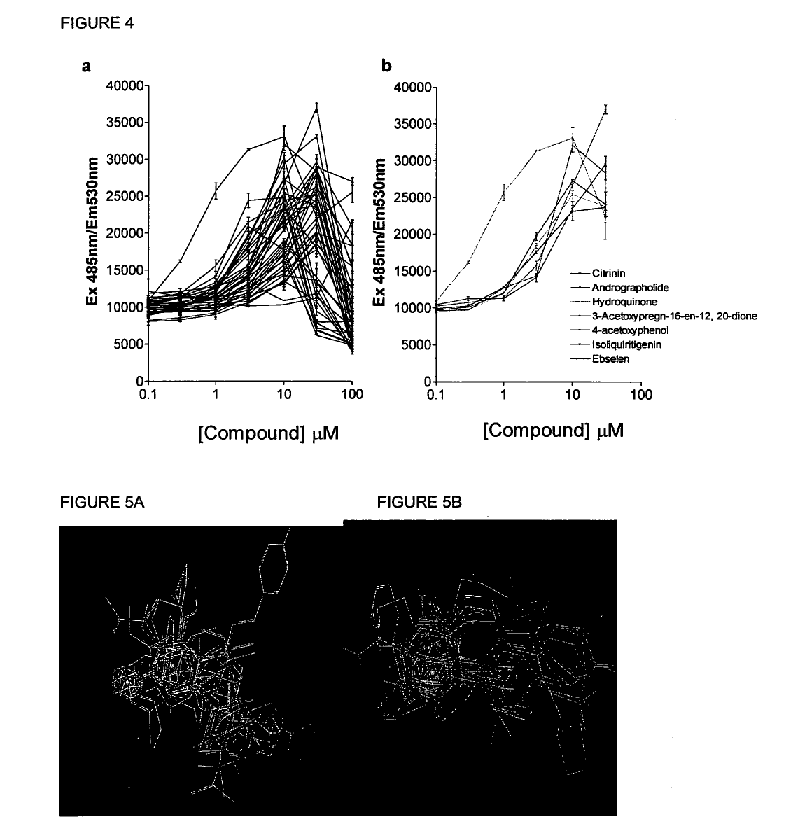

In order to screen the SPECTRUM collection of 2000 molecules the reporter assay was scaled down to a 384 well-plate format. To assess the suitability of the assay for library screening, a Z′ score calculation was performed by treating alternate wells with vehicle (0.1% DMSO) and 10 μM Ebselen as a positive control (see calculation in Methods). We have shown Ebselen gives a robust concentration response curve in this assay. The calculated Z′ score was 0.51 (FIG. 2b) which is acceptable for library screening. In addition, signal to noise (S/N) and signal to background (S/B) ratios were acceptable at 12.8 and 2.9 respectively. The library was subsequently screened at a single concentration per compound of 10 μM. Drug library dilutions and plating were carried out by a Q-BOT liquid handling system and both the 4×ARE-TK-GFP reporter cell line and TK-GFP control cell line were tested for their response to the compounds. An example set of data for the ARE-TK-GFP cell line from a single 384 well plate is shown in FIG. 3. Hits were identified as having data points more than three standard deviations above the background level, which was the average value of 24 wells treated with vehicle (0.1% DMSO) only. Hit compounds were checked to see if they generated a response in the control cell line due to either non-specific activation of transcription or autofluorescence of the compounds. In addition, any compounds showing signs of toxicity by enhancing ethidium homodimer fluorescence were excluded. The library screen was repeated with the 4×ARE—TK-CHO cell line only and compounds which emerged as hits from both screens were taken forward for further assessment. A total of 46 compounds were identified on this basis. The next step was to determine the compound concentration required to give a 50% response (EC₅₀) for these 46 hit compounds. Each compound was subjected to a 7 point concentration response curve in duplicate wells. Many compounds showed a bell shaped dose response curve similar to that seen for standard ARE inducers such as tBHQ and EGCG, due to toxicity at higher concentrations. FIG. 4a shows all 46 dose response curves in the first assay. The majority of compounds exhibit a bell-shaped dose response curve with toxicity at higher concentrations and many also have a very narrow window of ARE induction. FIG. 4b shows a set of compounds with enhancement of reporter expression over a broader concentration range (>1 log unit) or with minimal toxicity at higher concentrations. The concentration response curves for all 46 hits were repeated and the average EC₅₀ and average maximum fold induction of GFP fluorescence measured. The lowest concentration which caused a toxic response was also noted and the data are summarised in Table 2 (presented herein after), together with a brief description of the known bioactivity of these compounds. The lowest dose at which toxicity was observed is also included in the table. Compounds are ranked according to activity in the reporter assay. The most potent ARE inducer was the natural product andrographolide, the only compound with a sub-micromolar EC₅₀ (740 nM), this compound comes from the natural product Andrographis paniculata, and is used widely in Chinese and Indian herbal medicine. Of the 26 other natural products, a further two have been used in man; securinine, a GABAA receptor antagonist and CNS stimulant and isoliquiritigenin, a component of liquorice root which is an aldose reductase inhibitor. The remaining 19 products were synthetic small molecules or derivatives and of these a total of six molecules were approved drugs. Two alkylating anti-neoplastic drugs (pipobroman and mechlorethamine) a dopamine agonist (apomorphine hydrochloride), a topical skin whitener (hydroquinone), a loop diuretic (ethacrynic acid) and a vasodilator (isoxsuprine hydrochloride). One of the synthetic small molecules had reached phase three clinical trials in stroke (Ebselen).

### EXAMPLE 3

The effects of Nrf2-ARE inducing hit compounds on oxidative stress induced by serum withdrawal in motor neuronal and astrocytic cells was investigated. Since the activation of this pathway may vary depending on the cell type, we then went on to screen how well these hit compounds could protect a motor neuronal cell line (NSC34 cells) and rat (C6) and human (1321N1) astrocyte cell lines from oxidative stress induced by serum withdrawal. The cell lines were pre-treated with hit compound at a range of concentrations for 24 hours to activate the NRF2-ARE pathway. The compound was then removed and the cells subjected to a six hour serum withdrawal to induce oxidative stress. The degree of oxidative stress was measured using dichlorofluorescein (DCF) fluorescence and the degree of protection is shown in Table 3 as percentage reduction in DCF fluorescence for each of the three cell lines. Where it was possible to fit a curve, the concentration required to give a half maximal effect (IC₅₀) is also quoted. In general, hit compounds were more likely to show protective effects in the astrocyte cell lines than in the motor neuronal cell line. Table 3 (presented herein after) gives the assay results for all compounds, ranked by activity in the motor neuronal cell line. Only 9/46 compounds reduced the oxidative stress DCF signal induced by serum withdrawal in NSC34 cells, 18/46 compounds had no effect in this assay and the remaining 17 compounds were pro-oxidant in this assay. In other words as opposed to decreasing the oxidative stress caused by serum withdrawal they contributed to it and increased DCF fluorescence. In contrast only one compound was pro-oxidant in the 1321 N1 astrocyte cell line and 29/46 reduced the DCF signal by 30% or more. For the C6 cell line no compounds were pro-oxidant and 32/46 compounds reduced the DCF signal by 30% or more.

### EXAMPLE 4

In order to rationalize the biological results obtained, a general pharmacophore for the compounds reported in Table 1 was investigated, using the Pharmacophore Elucidator implemented in MOE (Molecular Operating Environment) [Molecular Operating Environment (MOE 2007.09). Chemical Computing Group, Inc. Montreal, Quebec, Canada http://www.chemcomp.com]. Considering the 24 molecules with an EC₅₀ of less than 10 μM for induction of the NRF2-ARE pathway in the CHO 4× ARE-TK cell line (Table 2), upon alignment, 22 of them presented two common features: an aromatic/hydrophobic moiety and a hydrogen bond acceptor moiety (FIG. 5a). It should be noted that EGCG also possesses these structural features and positively matches the calculated pharmacophore. Furthermore, when the 1321N1 astrocyte oxidative stress assay results (Table 3) were used for constructing the pharmacophore with a 3 μM activity threshold, one additional common aromatic/hydrophobic feature was identified (FIG. 5b). This is consistent with known Nrf2 activators which may act by electrophilic attack of sulphydryl groups on KEAP1, the cytoplasmic Nrf2 regulator. These preliminary modelling data can be used to understand the structural requirements of potential activators of the Nrf2 ARE pathway and could be taken into consideration in the design of novel structures for this class of compounds.

### EXAMPLE 6

The physical/chemical properties of the compounds were assessed. In addition to screening the compounds for functional effects in relevant cell types we also used Pipeline Pilot, a chemi-informatics programme to calculate chemical/physical properties, also shown in Table 3. ALogP (log of the partition coefficient in octanol/water) and Molecular polar surface area (mPSA) are different measures of the lipophilicity of the compounds and allow crude prediction of likely CNS penetrance. For CNS penetrance an AlogP less than 4 but greater than 1, and a mPSA below 100 (ideally 80) are optimal for passive diffusion across the BBB. In addition, the Lipinski filter was used to identify the non-drug like molecules and four were excluded-(swietenolide-3-acetate, endecaphylin X, lobaric acid and euphol acetate).

To filter out unwanted molecules only those with a protective or neutral effect in the NSC34 oxidative stress assay (Table 3) were selected and known cytotoxic molecules excluded (pipobroman, chlordane, alachlor, propachlor). Applying the AlogP and mPSA criteria to the remaining 22 molecules left 17 molecules for further investigation. These molecules are highlighted in bold in Table 3 and are designated the ‘best hit’ molecules. In addition the minimum dose at which toxicity was observed is shown. NA, not applicable (insufficient data, no concentration response or no inhibition). For Table 3 compounds are sorted by protective capacity in the NSC34 cell line. ARE inducers were far more effective at protecting the astrocyte cell lines (1321 N1 and C6) from oxidative stress compared to NSC34 cells.

### EXAMPLE 6

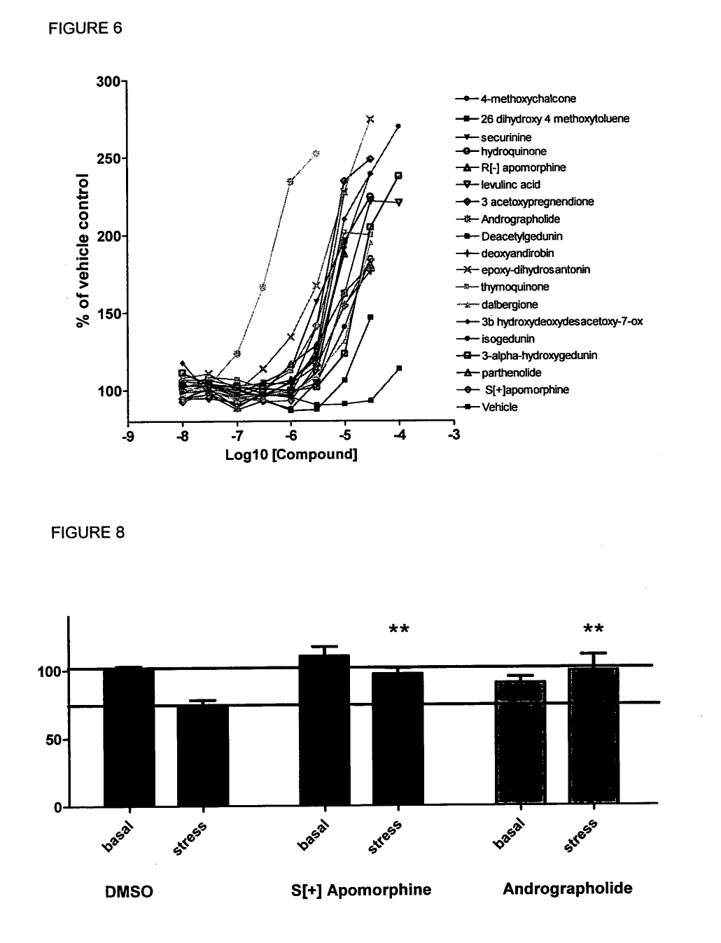

NRF2-ARE Inducing activity of the best hit compounds in neuronal and astrocytic cell lines was investigated. In order to determine whether the differences in protection in astrocytic and motor neuronal cell lines was due to differences in the degree of activation of the NRF2-ARE pathway in these cell types, the NRF2-ARE reporter construct was stably expressed in both the astrocytic (C6) and motor neuronal (NSC34) cell lines. The 17 best hit molecules were then screened in each cell line. We also screened the S [+] enantiomer of apomorphine as it can exist as either an R[−] or S[+] enantiomer. The R[+] enantiomer has dopamine agonist activity whereas the S[+] enantiomer has lost this activity and so we wanted to determine whether it retains NRF2-ARE inducing activity. The results for the C6 reporter cell line are shown in FIG. 6. In general activation of the NRF2-ARE pathway in the C6 cells was similar to that seen in the CHO cell line. The NSC34 reporter cell line showed minimal if any activation with the same set of concentration response curves suggesting the underlying cause for greater protection against oxidative stress in astrocytic cell lines versus the NSC34 cells seen earlier was due to a much more robust activation of the NRF2-ARE pathway in astrocyte cell lines. In addition, the S[+] enantiomer of apomorphine was equally potent in terms of NRF2-ARE activation compared with the R[+] enantiomer indicating that this activity is unrelated to agonism of dopamine receptors as the S[+] enantiomer is not a dopamine agonist.

It was an objective to identify molecules with potential to be fast-tracked for clinical testing in Motor Neurone Disease. One of the key criteria in this regard was that the molecules have a history of use in man albeit not for MND. Of the 17 best hit molecules, two had a history of use in man as natural products (securinine, andrographolide) and one was a currently approved drug used for the acute rescue of ‘off’ episodes in Parkinsons Disease (apomorphine hydrochloride as the R enantiomer). In addition, hydroquinoine has a history of use, although as a topically applied skin whitener. Of these drugs andrographolide and the S[+] enantiomer of apomorphine were selected for further assessment as securinine has well described convulsant properties in mice and man and the lack of dopamine agonism in the S[+] enantiomer of apomorphine was considered a distinct advantage.

### EXAMPLE 7

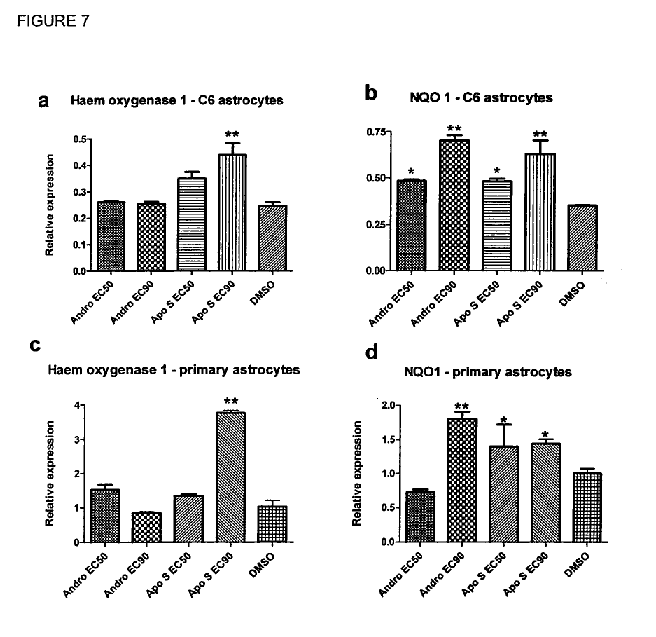

Induction of ARE target gene expression in C6 cells and primary mouse astrocytes by andrographolide and S[+] apomorphine was investigated. To confirm that these preferred or ‘lead’ molecules were able to activate the NRF2-ARE pathway leading to target gene expression in astrocytes, a multiplex RT-PCR assay was developed on the GenomeLab™ GeXP genetic analysis system for 9 genes of interest. C6 cells were treated for 24 h with Andrographolide and S[+] Apomorphine at EC₅₀ and EC₉₀ concentrations as determined in C6-4×ARE-TK reporter cells. Only two genes, Haem oxygenase 1 and NQO1, showed statistically significant changes in gene expression, which was confirmed by standard quantitative RT-PCR (FIGS. 7A and 7B).

### EXAMPLE 8

Since the lead NRF2 inducing molecules were able to increase expression of target genes in primary mouse motor neurones, co-cultures consisting of primary mouse motor neurones (MN) on an astrocyte feeder layer were exposed to an oxidative insult following pre-treatment with either Andrographolide of [S+] apomorphine (FIG. 8). The co-cultures were then challenged for 6 hours with 10 μM menadione to induce oxidative stress and motor neurones stained and counted. In DMSO control cells an approximately 25% reduction in motor neurone number was observed which was not seen in wells treated with either drug. These results indicate that NRF2 inducers protect motor neurones (MN) from oxidative stress in primary mouse astrocyte/MN co-cultures.

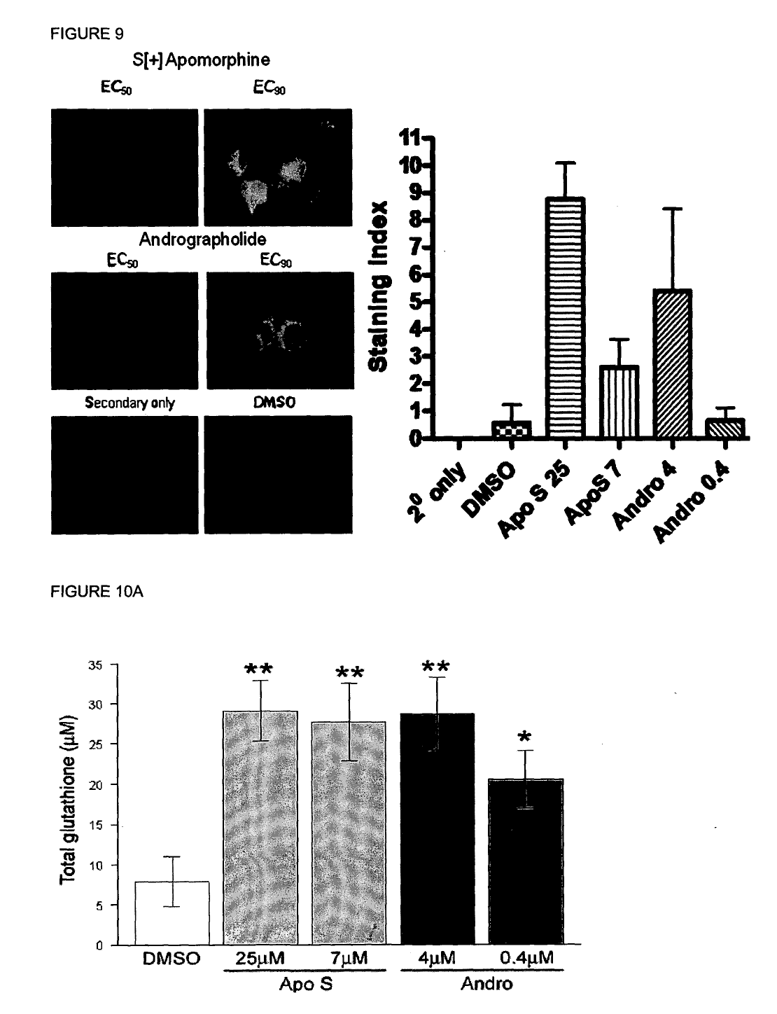

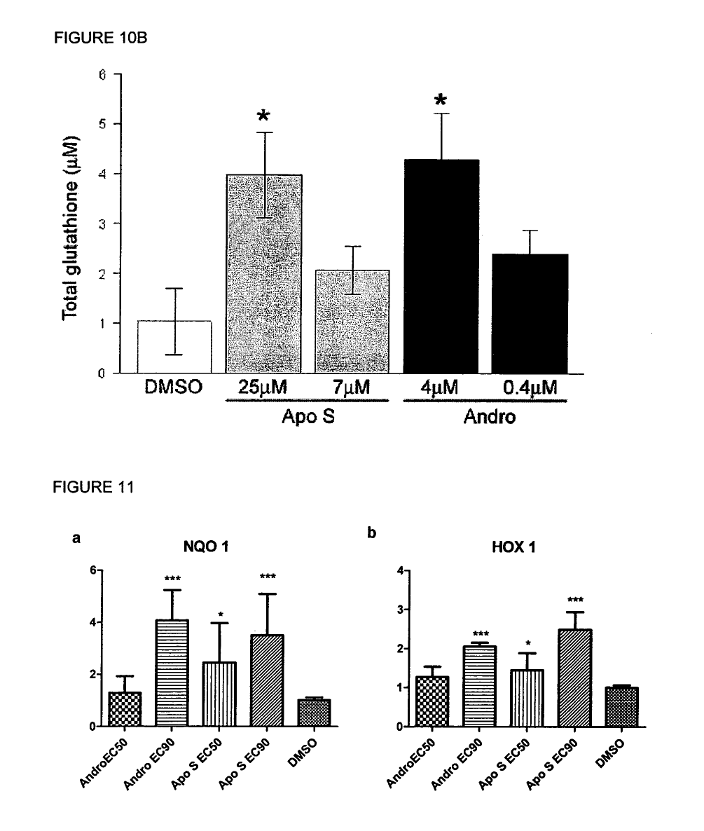

Standard quantitative RT-PCR of the NQO1 and HO-1 genes in primary mouse astrocytes under the same conditions also evinced significant increases in gene expression (FIGS. 7C and 7D). In order to confirm that changes in gene expression lead to concomitant increases in protein expression, primary mouse astrocytes were assessed for elevated levels of HO-1 by immunofluoresence staining (FIG. 9) which confirmed a dose-dependent enhancement of expression over that seen in DMSO treated cells. We also examined a functional effect of this enhanced anti-oxidant capacity by examining total glutathione levels under the same conditions in both the primary astrocytes themselves and in the media collected from treated astrocytes (FIG. 10). This showed that glutathione levels were elevated in both the cells themselves and in media—providing one mechanism by which Nrf2 responsive astrocytes may protect neighbouring non-ARE responsive MNs from oxidative challenge.

### EXAMPLE 9

In an attempt to rationalize the biological results obtained, we have tried do identify a general pharmacophore for the compounds reported in Table 2, using the Pharmacophore Elucidator implemented in MOE (Molecular Operating Environment) [Molecular Operating Environment (MOE 2007.09). Chemical Computing Group, Inc. Montreal, Quebec, Canada http://www.chemcomp.com]. Considering the 24 molecules with an EC50 of less than 10 μM for induction of the NRF2-ARE pathway in the CHO ARE-TK cell line (Table 2), upon alignment, 22 of them presented two common features: an Aromatic/Hydrophobic moiety and a Hydrogen Bond Acceptor moiety (FIG. 4a). It should be noted that EGCG also possesses these structural features and positively matches the calculated pharmacophore. Furthermore, when the 1321N1 astrocyte oxidative stress assay results (Table 3) were used for constructing the pharmacophore, using a 3 μM activity threshold, one extra common Aromatic/Hydrophobic feature was identified (FIG. 5b). This is consistent with known NRF2 activators which may act by nucleophilic attack of sulphydryl groups on KEAP1, the cytoplasmic NRF2 regulator. These preliminary modelling data can be used to understand the structural requirements of potential activators of the NRF2-ARE pathway and could be taken in consideration in the design of novel structures for this class of compounds.

### EXAMPLE 10

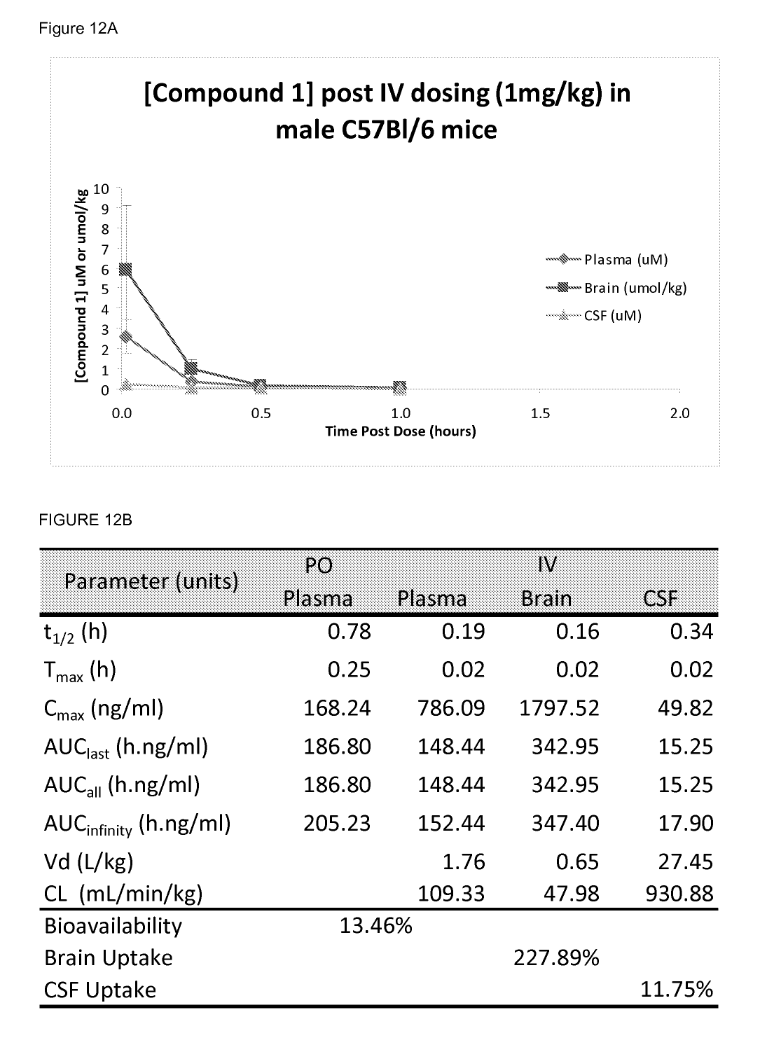

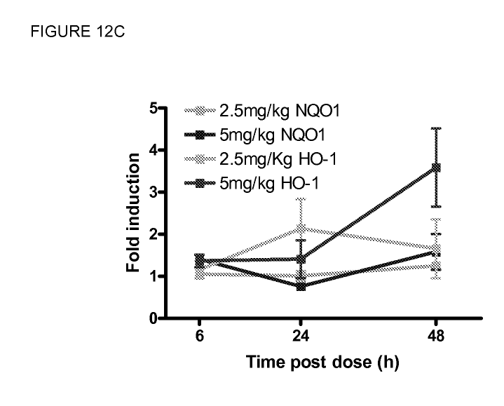

Since previous work had demonstrated an attenuated Nrf2 response in motor neuronal cell lines expressing mutant SOD and in post mortem material in astrocytes from familial human SOD1 cases we investigated whether our lead inducers could still activate Nrf2 in astrocytes expressing G93A mutant SOD1. It was first determined whether the Nrf2 regulated genes NAD(P)H:quinine oxidoreductase (NQO1) and heme oxygenase 1 (HOX1) could be induced in primary mouse astrocytes from G93A mutant SOD1 transgenic mice (FIG. 11). Quantitative RT-PCR demonstrated a significant increase in transcripts for NQO1 and HO1 following a 24 hour pre-treatment with Andrographolide and S[+] Apomorphine at their EC₉₀ concentrations (FIG. 11).

### EXAMPLE 11

FIG. 12 shows the in vivo pharmacokinetics and pharmacodynamics of [S+] apomorphine in male C57BI/6 mice. Following a single intravenous dose of [S+] apomorphine levels of the compound were detected in plasma, brain and cerebral spinal fluid (FIGS. 12A and 12B) and following subcutaneous doses there was significant induction at 24 hours post dose of HO-1 and NQO-1 transcriptsas determined by QRT-PCR (FIG. 12C). Accordingly, [S+] apomorphine can lead to a prolonged increase in expression of anti-oxidant enzymes through transcriptional activation.

These data demonstrate that several compounds have been identified that are NF2-ARE pathway activators in vitro and that they may also activate this pathway in vivo.

TABLE 2

| | | ARE reporter assay | | |
|---|---|---|---|---|
| | | Average | | Lowest |
| | | fold GFP | | dose |
| | | induction at | Average | with |
| | | maximal | EC50 | toxicity |
| Name | Bioactivity and/or source | response | (μM) | (μM) |
| Andrographolide | Natural product used in Indian | 3.61 | 0.74 | 100 |
| (diterpenoid) | and Chinese Medicine | | | |
| deoxygedunin | Similar to known anti-malarial | 1.82 | 1.15 | 30 |
| (triterpenoid) | | | | |
| Propachlor | pesticide, high dose neurotoxic | 3.25 | 1.49 | 100 |
| Securinine | GABAA receptor antagonist, | 3.00 | 2.12 | 30 |
| | alkaloid, CNS stimulant | | | |
| Dalbergione | Natural product | 2.09 | 2.47 | 30 |
| Isoliquintigenin | Licorice flavanoid, | 2.58 | 2.52 | 30 |
| | aldose reductase inhibitor | | | |
| 4 acetoxyphenol | Natural product, antioxidant | 2.78 | 2.91 | 100 |
| 3 acetoxypregn-16- | Natural product derivative | 3.14 | 2.98 | 30 |
| en-12,20-dione | | | | |
| 2 methyl gramine | Natural product derivative | 2.27 | 3.00 | 100 |
| hydroquinone | skin whitener, antioxidant | 2.95 | 3.07 | 100 |
| (1,4 dihydroxybenzene) | | | | |
| Alachlor | herbicide, high dose neurotoxic | 3.50 | 3.29 | 100 |
| kinetin riboside | Purine derivative | 1.97 | 3.39 | 30 |
| Deacetylgedunin | Natural Product | 3.05 | 3.50 | 30 |
| sappanone A trimethyl ether | Natural product derivative | 1.99 | 3.50 | 100 |
| swietenolide 3 acetate | possibly natural? Liminoids | 2.82 | 3.50 | 100 |
| | are anti fungal | | | |
| 4 hydroxychalcone | flavonol, glutathione reductase | 1.88 | 3.53 | None |
| | inhibitor, plant polyphenol | | | |
| Ebselen | Antioxidant | 2.81 | 4.00 | 100 |
| Citrinin | Natural product, antibacterial | 3.26 | 4.10 | 30 |
| Mechloroethamine | antineoplastic, alkylating agent | 2.25 | 5.50 | 30 |
| Isoxsuprine hydrochloride | Vasodilator | 1.52 | 8.70 | 100 |
| 3β hydroxydeoxydesacetoxy- | Natural Product | 2.55 | 8.71 | 30 |
| 7-oxogedunin | | | | |
| Chlordane | pesticide, high dose neurotoxic | 1.56 | 9.18 | 100 |
| 3 methylorsellinic acid | Benzoic derivative, | 2.87 | 9.59 | 100 |
| | Phospholipase A2 inhibitors | | | |
| Pipobroman | antineoplastic, alkylating agent | 3.21 | 10.0 | 100 |
| Deoxyandirobin lactone | Semisynthetic | 3.03 | 11.1 | 100 |
| (similar structure) | | | | |
| levulinc acid | Natural product derivative | 2.94 | 12.0 | None |
| Ethacrynic acid | Diuretic | 2.72 | 14.0 | 100 |
| penicillic acid | fungal toxin, antibacterial | 2.64 | 14.9 | 100 |
| 4 methoxychalcone | Natural product | 2.13 | 15.0 | 100 |
| 3 methoxycatechol (similar | Oncogenic in rats | 3.28 | 15.4 | 100 |
| to hydroquinone) | | | | |
| Lobaric acid | Natural product | 2.53 | 17.0 | 100 |
| epoxy(4,5α)- | anti parasitic, toxicity affects vision | 2.91 | 18.2 | 100 |
| 4,5-dihydrosantonin | | | | |
| endecaphyllin X | nitrogenous glucoside miserotoxin | 2.15 | 18.8 | None |
| | (neurotoxic and respiratory toxin) | | | |
| Benzyl Isothiocyanate | Natural product, antineoplastic, | 1.81 | 24.0 | None |
| | antibacterial, antifungal | | | |
| Apomorphine hydrochloride | impotence drug and PD | 1.90 | 25.9 | 100 |
| | dopamine agonist, known CNS | | | |
| 2,6-dihydroxy- | delivery | 3.05 | 45.0 | 100 |
| 4-methoxytoluene | | | | |
| Parthenolide | NSAID, HCV inhibitors | 2.77 | 136 | 100 |
| Isogedunin | Natural Product | 3.11 | 550 | None |
| methyl-7- | synthetic analog antioxidant | 2.83 | 2054 | 100 |
| deshydroxypyrogallin- | | | | |
| 4-carboxylate | | | | |
| 4-Methoxydalbergione | Natural product | 2.11 | ?/11 | 100 |
| Antiarol | Natural product | 1.64 | 1.5/23 | 100 |
| Thymoquinone | Natural product | 2.58 | 12/1.26 | 30 |
| Euphol acetate | Natural product | 2.20 | 99? | 100 |
| Retusoquinone | Natural product | 2.04 | NC/35? | 100 |
| 3-alpha-hydroxygedinin | Natural product | 3.01 | one | 100 |
| | | | dose | |
| | | | effect | |

TABLE 3

| | NSC34 oxidative | | | 1321N1 oxidative | | | C6 oxidative | | | | |
|---|---|---|---|---|---|---|---|---|---|---|---|
| | stress assay | | | stress assay | | | stress assay | | | | |
| | | Maximal % | Lowest | | Maximal % | Lowest | | Maximal % | Lowest | | |
| | | reduction in | dose with | | reduction in | dose with | | reduction in | dose with | Calculated | |
| | IC50 | Oxidative | toxicity | IC50 | Oxidative | toxicity | IC50 | Oxidative | toxicity | properties | |
| Name | (μM) | stress | (μM) | (μM) | stress | (μM) | (μM) | stress | (μM) | ALogP | mPSA |
| Pipobroman | NA | 30 | none | 3.16 | 19 | 10 | 11 | 71 | none | 0.647 | 40.62 |
| 2,6-Dihydroxy-4- | ~3.26 | 28 | none | NA | 40 | none | 5.29 | 16 | 10 | 1.815 | 49.68 |
| methoxytoluene | | | | | | | | | | | |
| Apomorphine | 0.174 | 25 | 10 | 1.7 | 79 | none | 1.69 | 62 | none | 3.498 | 43.7 |
| hydrochloride | | | | | | | | | | | |
| 4-Methoxychalcone | NA | 24 | none | 3.6 | 30 | none | 3.55 | 82 | none | 3.685 | 26.3 |
| Securinine | NA | 20 | none | 18.3 | 55 | none | 0.645 | 67 | none | 1.448 | 29.53 |
| Levulinic acid | NA | 15 | none | 3.31 | 58 | none | 7.69 | 62 | none | 1.652 | 54.37 |
| 3-Alpha- | NA | 14 | 10 | NA | 25 | none | 3.22 | 42 | none | 3.83 | 85.97 |
| hydroxygedinin | | | | | | | | | | | |
| Penicillic acid | NA | 9 | none | NA | 45 | none | 4.62 | 68 | none | 0.778 | 55.76 |
| Deoxygedunin | NA | 7 | 10 | 1.41 | 25 | 10 | 2.19 | 61 | none | 4.217 | 82.81 |
| Hydroquinone | NA | 0 | none | 3.38 | 80 | none | 7.07 | 67 | none | 1.346 | 40.46 |
| Andrographolide | NA | 0 | 10 | 0.501 | 35 | 10 | 0.192 | 69 | 10 | 2.056 | 86.99 |
| Deacetylgedunin | NA | 0 | none | 2.37 | 28 | 10 | 5.36 | 70 | none | 2.97 | 89.26 |
| 3β Hydroxy- | NA | 0 | 3 | 3.96 | 62 | none | 3.07 | 67 | none | 2.683 | 89.26 |
| deoxydesacetoxy-7- | | | | | | | | | | | |
| oxogedunin | | | | | | | | | | | |
| Isogedunin | NA | 0 | 1 | NA | 10 | none | NA | | | 3.349 | 95.34 |
| Deoxyandirobin | NA | 0 | none | 1.64 | 65 | none | 2.87 | 38 | none | 3.399 | 85.97 |
| lactone | | | | | | | | | | | |
| Endecaphyllin X | NA | 0 | 10 | NA | 56 | none | 3.15 | 40 | none | −9.6E−02 | 317.93 |
| Parthenolide | NA | 0 | 1 | 28.4 | 47 | none | 3.11 | 81 | none | 2.923 | 38.82 |
| Alachlor | NA | 0 | 10 | 1.97 | 83 | none | 6.22 | 62 | none | 3.425 | 29.54 |
| Propachlor | NA | 0 | 10 | 0.829 | 50 | 10 | 0.645 | 74 | 10 | 2.415 | 20.31 |
| Chlordane | NA | 0 | 3 | NA | 68 | none | 3.57 | 20 | none | 4.97 | 0 |
| Epoxy(4,5α)-4,5- | NA | 0 | 3 | 0.662 | 63 | none | 2.13 | 62 | none | 1.598 | 55.89 |
| dihydrosantonin | | | | | | | | | | | |
| Kinetin riboside | NA | 0 | 3 | 0.246 | 25 | 3 | 3.84 | 40 | 10 | −1.033 | 138.68 |
| 3 Acetoxypregn-16- | NA | 0 | none | 12.4? | 75 | none | 5.34 | 50 | none | 3.213 | 60.44 |
| en-12,20-dione | | | | | | | | | | | |
| Thymoquinone | NA | 0 | none | 11 | 25 | none | 0.014 | 32 | none | 2.288 | 34.14 |
| Citrinin | NA | 0 | none | 0.338 | 28 | none | 20 | 23 | none | 0.928 | 83.83 |
| Dalbergione | NA | 0 | none | 64 | 38 | none | 46 | 40 | none | 2.94 | 34.14 |
| Swietenolide 3 | NA | 0 | 0.3 | NA | 60 | none | 4.22 | 50 | none | 2.633 | 129.34 |
| acetate | | | | | | | | | | | |
| Isoliquiritigenin | NA | −6 | 1 | 1.78 | 50 | none | 28? | 46 | none | 2.976 | 77.76 |
| Mechloroethamine | NA | −10 | 10 | 0.302 | 37 | 10 | 3.42 | 67 | none | 1.65 | 3.24 |
| 3-Methylorsellinic | NA | −12 | 0.3 | 3.26 | 53 | none | 3.1 | 56 | none | 1.948 | 77.76 |
| acid | | | | | | | | | | | |
| 3-Methoxycatechol | NA | −13 | 0.3 | NA | 39 | none | 1.2 | 46 | 10 | 1.329 | 49.68 |
| Isoxsuprine | NA | −16 | none | NA | ? | none | 23 | 40 | none | 3.384 | 61.71 |
| hydrochloride | | | | | | | | | | | |
| Methyl-7- | NA | −23 | 0.3 | NA | 45 | none | 2.95 | 70 | none | 1.133 | 104.06 |
| deshydroxy- | | | | | | | | | | | |
| pyrogallin-4- | | | | | | | | | | | |
| carboxylate | | | | | | | | | | | |
| Retusoquinone | NA | −25 | none | | 0 | none | 2.06 | 15 | none | 2.684 | 17.07 |
| Lobaric acid | NA | −26 | none | | 0 | none | 3.14 | 9 | none | 5.993 | 119.36 |
| Benzyl | NA | −28 | none | NA | 8 | none | 3.47 | 27 | none | 2.596 | 44.45 |
| isothiocyanate | | | | | | | | | | | |
| Ethacrynic acid | NA | −30 | 10 | 3.01 | 25 | none | 3.01 | 31 | none | 3.964 | 63.6 |
| 2-Methyl gramine | NA | −30 | 3 | 11 | 36 | none | 32 | 36 | none | 2.342 | 19.03 |
| Euphol acetate | NA | −43 | none | 18 | 38 | none | NA | 0 | none | 8.498 | 26.3 |
| 4-Methoxy- | NA | −44 | none | NA | 43 | none | 2.5 | 14 | none | 2.627 | 43.37 |
| dalbergione | | | | | | | | | | | |
| Antiarol | NA | −50 | 10 | NA | 35 | none | 3.05 | 23 | 10 | 1.538 | 47.92 |
| Sappanone A | NA | −60 | none | 0.279 | 40 | none | NA | 27 | none | 3.35 | 53.99 |
| trimethyl ether | | | | | | | | | | | |
| 4-Acetoxyphenol | NA | −70 | none | 0.298 | 42 | none | NA | 0 | none | 1.355 | 46.53 |
| Ebselen | NA | −80 | none | 0.193 | 20 | none | NA | 30 | none | 3.227 | 20.31 |
| 4-Hydroxychalcone | | | | | | | | | | 3.459 | 37.29 |

1-7. (canceled)

8. A method for treating an individual suffering from motor neurone disease, comprising administering to said individual a therapeutically effective amount of a therapeutic agent, the therapeutic agent being an Nrf2-ARE pathway activator selected from the group consisting of andrographolide, S [+] apomorphine, and variations, derivatives and substitutions thereof.

9. The method of claim 8, wherein the motor neurone disease is selected from a group consisting of amyotrophic lateral sclerosis (ALS), primary lateral sclerosis (PLS), progressive muscular atrophy (PMA) and progressive Bulbar palsy (PBP).

10. A method for treating an individual suffering from a condition selected from the group consisting of Huntington's disease, age related macular degeneration, photogenic oxidative stress and radiation-induced cell damage, comprising administering to said individual a therapeutically effective amount of a therapeutic agent, the therapeutic agent being an Nrf2-ARE pathway activator selected from the group consisting of andrographolide, S [+] apomorphine, and variations, derivatives and substitutions thereof.

11. A method for preserving an organ in a transplant/surgical procedure, comprising contacting the organ with a therapeutic agent, the therapeutic agent being an Nrf2-ARE pathway activator selected from the group consisting of andrographolide, S [+] apomorphine, and variations, derivatives and substitutions thereof.

12. A method for stabilisation of a cell culture, comprising contacting the cell culture with a therapeutic agent, the therapeutic agent being an Nrf2-ARE pathway activator selected from the group consisting of andrographolide, S [+] apomorphine, and variations, derivatives and substitutions thereof.

13. The method of claim 8, wherein the agent is andrographolide or a derivative thereof of the formula 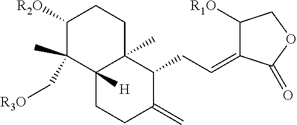
where R₁, R₂ and R₃ independently represent hydrogen, acyl, phenyl, mono- or polyphosphate, mono- or polysulfate, glycosyl, cyclic or acyclic alkyl, alkenyl or alkynyl, wherein said phosphate or sulfate derivative is in the form of a free acid or a salt.

14. The method of claim 8, wherein the agent is andrographolide. (New) The method of claim 10, wherein the agent is andrographolide or a derivative thereof of the formula 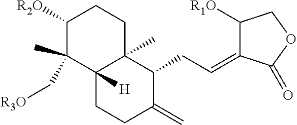
where R₁, R₂ and R₃ independently represent hydrogen, acyl, phenyl, mono- or polyphosphate, mono- or polysulfate, glycosyl, cyclic or acyclic alkyl, alkenyl or alkynyl, wherein said phosphate or sulfate derivative is in the form of a free acid or a salt.

16. The method of claim 10, wherein the agent is andrographolide.

17. The method of claim 11, wherein the agent is andrographolide or a derivative thereof of the formula 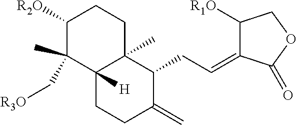
where R₁, R₂ and R₃ independently represent hydrogen, acyl, phenyl, mono- or polyphosphate, mono- or polysulfate, glycosyl, cyclic or acyclic alkyl, alkenyl or alkynyl, wherein said phosphate or sulfate derivative is in the form of a free acid or a salt.

18. The method of claim 11, wherein the agent is andrographolide.

19. The method of claim 12, wherein the agent is andrographolide or a derivative thereof of the formula 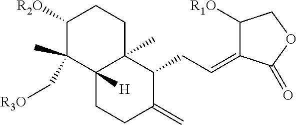
where R₁, R₂ and R₃ independently represent hydrogen, acyl, phenyl, mono- or polyphosphate, mono- or polysulfate, glycosyl, cyclic or acyclic alkyl, alkenyl or alkynyl, wherein said phosphate or sulfate derivative is in the form of a free acid or a salt.

20. The method of claim 12, wherein the agent is andrographolide.

21. The method of claim 8, wherein the therapeutic agent is administered by a route selected from the group consisting of parenteral, intravenous, intramuscular, intraperitoneal, subcutaneous administration, and direct delivery into a target organ or tissue by injection or infusion.

22. The method of claim 10, wherein the therapeutic agent is administered by a route selected from the group consisting of parenteral, intravenous, intramuscular, intraperitoneal, subcutaneous administration, and direct delivery into a target organ or tissue by injection or infusion.


SEQUENCE LISTING (25 sequences)

SEQ ID NO: 1 - DNA, 41 bp, Artificial
  OLIGONUCLEOTIDE PRIMER
tagcttggaaatgacattgctaatcgtgacaaagcaacttt

SEQ ID NO: 2 - DNA, 20 bp, Artificial
  OLIGONUCLEOTIDE PRIMER
aagaggctaagaccgccttc

SEQ ID NO: 3 - DNA, 21 bp, Artificial
  OLIGONUCLEOTIDE PRIMER
tgtctgtgagggactctggtc

SEQ ID NO: 4 - DNA, 21 bp, Artificial
  OLIGONUCLEOTIDE PRIMER
cctgaatgagcaggtgaaatc

SEQ ID NO: 5 - DNA, 20 bp, Artificial
  OLIGONUCLEOTIDE PRIMER
cagggtgtgcttgtcaaaga

SEQ ID NO: 6 - DNA, 20 bp, Artificial
  OLIGONUCLEOTIDE PRIMER
acgacgtggagacagagacc

SEQ ID NO: 7 - DNA, 20 bp, Artificial
  OLIGONUCLEOTIDE PRIMER
aaaagtgtggccatcgtagc

SEQ ID NO: 8 - DNA, 20 bp, Artificial
  OLIGONUCLEOTIDE PRIMER
aaaggcctctaagccagacc

SEQ ID NO: 9 - DNA, 20 bp, Artificial
  OLIGONUCLEOTIDE PRIMER
gcagatagtggccaactggt

SEQ ID NO: 10 - DNA, 20 bp, Artificial
  OLIGONUCLEOTIDE PRIMER
agacagccgcatcttcttgt

SEQ ID NO: 11 - DNA, 20 bp, Artificial
  OLIGONUCLEOTIDE PRIMER
ttgatggcaacaatgtccac

SEQ ID NO: 12 - DNA, 20 bp, Artificial
  OLIGONUCLEOTIDE PRIMER
aaacggctaccacatccaag

SEQ ID NO: 13 - DNA, 20 bp, Artificial
  OLIGONUCLEOTIDE PRIMER
cctccaatggatcctcgtta

SEQ ID NO: 14 - DNA, 20 bp, Artificial
  OLIGONUCLEOTIDE PRIMER
cagcatacagcaggacatgg

SEQ ID NO: 15 - DNA, 20 bp, Artificial
  OLIGONUCLEOTIDE PRIMER
cagtgaggggatcgatgagt

SEQ ID NO: 16 - DNA, 20 bp, Artificial
  OLIGONUCLEOTIDE PRIMER
cgcccatagactttttgagg

SEQ ID NO: 17 - DNA, 20 bp, Artificial
  OLIGONUCLEOTIDE PRIMER
catttcgtcttcctcgtggt

SEQ ID NO: 18 - DNA, 20 bp, Artificial
  OLIGONUCLEOTIDE PRIMER
accttgctttccatcaccac

SEQ ID NO: 19 - DNA, 20 bp, Artificial
  OLIGONUCLEOTIDE PRIMER
tctccagacgcttcttccac

SEQ ID NO: 20 - DNA, 20 bp, Artificial
  OLIGONUCLEOTIDE PRIMER
ttgctgacaggatgcagaag

SEQ ID NO: 21 - DNA, 20 bp, Artificial
  OLIGONUCLEOTIDE PRIMER
aagggtgtaaaacgcagctc

SEQ ID NO: 22 - DNA, 20 bp, Artificial
  OLIGONUCLEOTIDE PRIMER
tggccatgtcctatgtgaaa

SEQ ID NO: 23 - DNA, 20 bp, Artificial
  OLIGONUCLEOTIDE PRIMER
aaggggttgggaaagatgag

SEQ ID NO: 24 - DNA, 20 bp, Artificial
  OLIGONUCLEOTIDE PRIMER
gggcttgacatccacttcat

SEQ ID NO: 25 - DNA, 21 bp, Artificial
  OLIGONUCLEOTIDE PRIMER
cccgccttgaatgtagaattt